(19) United States
(12) Patent Application Publication
(10) Pub. No.: US 2025/0082482 A1
(43) Pub. Date: Mar. 13, 2025

(54) BALLOON CATHETERS

(71) Applicant: Ridgeback Technologies Ltd., Galway (IE)

(72) Inventor: Raphael Blowick, Galway (IE)

(21) Appl. No.: 18/266,245

(22) PCT Filed: Dec. 7, 2021
(86) PCT No.: PCT/EP2021/084654

§ 371 Date: Jun. 8, 2023

(30) Foreign Application Priority Data
Dec. 8, 2020 (GB) 2019332.2

(51) Int. Cl. A61F 2/958; A61M 25/10

(52) U.S. Cl. CPC A61F 2/958; A61M 25/1002

(57) ABSTRACT

A balloon for a perfusion balloon catheter may include a flexible envelope and a spine structure arranged within the envelope. The envelope may define at least two lobes that are disposed mutually spaced angularly about a central longitudinal axis and that project radially, when the envelope is expanded under internal fluid pressure, to define a plurality of longitudinal perfusion channels outside the envelope between the at least two lobes. The spine structure may extend outwardly with respect to the central longitudinal axis to join the envelope at a plurality of junctions disposed angularly between the at least two lobes in alignment with the plurality of perfusion channels. The spine structure may define a longitudinally-extending spine channel that surrounds the central longitudinal axis.

This invention relates to balloon catheters for deployment in body conduits, such as vessels or passageways of the heart, where it may be necessary to provide for fluids such as blood to flow past the deployed catheter. For this purpose, the invention relates to a perfusion balloon that has a lobed cross-section when inflated, for example a bi-lobal or tri-lobal cross-section, creating longitudinal flow paths around the balloon in parallel channels defined between the lobes. The invention is mainly, but not exclusively, intended for structural heart and oesophageal applications.

The invention relates particularly to dilation catheters for use in relieving constriction of blood flow caused by a stenosis in, for example, a heart valve or a coronary artery. An example of the use of such catheters is in coronary angioplasty procedures, which involve navigating a balloon catheter through the patient's vasculature to a stenosis and then injecting a fluid into the balloon under elevated pressure to inflate it. The expanding balloon presses the stenosis radially outwardly and compresses it against the wall of the blood vessel to increase the effective cross-sectional area of the vessel and so to restore an acceptable rate of blood flow. The balloon may act directly against the stenosis or via an expandable endoprosthesis or stent surrounding the balloon, which implant or graft may then remain in situ after the balloon has been deflated and withdrawn from the vasculature.

Complete occlusion of a coronary artery can only be tolerated momentarily in view of the risk of damage to tissue that relies on blood flow through that vessel. Consequently, perfusion balloon catheters have been developed to allow blood to continue flowing along channels that extend through or around the inflated balloon. Examples of such catheters are described in U.S. Pat. No. 4,581,017 and U.S. Pat. No. 5,087,247.

It is well known for the balloon of a balloon catheter to have an undulating or lobed cross-section when in a collapsed state. Primarily, the purpose of such a cross-section has been to optimise the compactness of the collapsed balloon to ease its travel through the vasculature to and from the site of a lesion. For example, U.S. Pat. No. 6,544,224 discloses a balloon catheter with a balloon that defines at least two lobes when in an uninflated configuration and a cylindrical surface when in an inflated configuration. Each lobe forms a deflated balloon wing when the balloon is deflated. The balloon may have a uniform or non-uniform wall thickness.

Similarly, U.S. Pat. No. 5,087,246 discloses a balloon dilatation catheter with a fluted balloon that defines at least three wings. When the balloon collapses, it assumes a fluted, lower-profile configuration that is better adapted to pass through narrow channels, such as in endoscopes and guide catheters.

Longitudinal fluting has also been proposed in perfusion balloon catheters to define flow paths around an expanded balloon. For example, U.S. Pat. No. 5,108,370 discloses a perfusion balloon catheter for angioplasty that has a balloon formed so that, when inflated, one or more channels are provided for the flow of bodily fluids or blood past the inflated balloon. In one embodiment, the balloon defines a multiple-lobed cross-section that allows fluids to flow along channels defined between the lobes.

EP 0737488 discloses a dilation catheter comprising an expandable balloon that has a number of relatively stiff sections extending longitudinally and relatively pliable sections between the stiffer sections, also extending longitudinally. Those relatively pliable sections form lobes when the balloon is in an expanded state. Similarly, U.S. Pat. No. 5,792,300 discloses a dilation catheter including a dilation balloon with a compliant portion that bulges radially outwardly when the balloon is expanded and a non-compliant longitudinal section that traverses the length of the balloon and does not extend radially outwardly to the same extent when the balloon is in its expanded condition, hence forming a perfusion channel.

U.S. Pat. No. 5,501,667 discloses a dilation catheter whose balloon contains a longitudinally extending septum that divides the balloon into a plurality of chambers. The septum crosses a central longitudinal axis of the balloon. A guidewire lumen is offset relative to the septum and the central longitudinal axis.

Another approach to defining perfusion channels is taught by the ‘GORE® Tri-Lobe balloon catheter’ supplied by W. L. Gore & Associates, Inc. of Arizona, USA. In that example, three polyurethane balloons are mounted in parallel on the distal end of a multi-lumen catheter shaft. Each of three inflation lumens of the shaft communicates with a respective one of the balloons. A common inflation port at a proximal end of the shaft communicates with all of the inflation lumens. Additionally, a guidewire lumen allows introduction of a guidewire for over-the-wire access. The three expanded balloons come into contact with, and press outwardly against, the surrounding wall of the vessel or implant, hence exerting dilatation force on three axes angularly spaced by 120 degrees while blood continues to flow around the balloons.

The balloon configurations taught by EP 0737488, U.S. Pat. Nos. 5,792,300, 5,501,667 and the GORE® Tri-Lobe balloon catheter suffer from various drawbacks that the invention seeks to address. For example, all-and especially the GORE® product-are complex and are not optimally compact when collapsed.

The relatively compliant and non-compliant sections proposed by EP 0737488 and U.S. Pat. No. 5,792,300 do not provide the inflated balloon with optimal dimensional accuracy and stability. Consequently, the cross-sectional shape and area of their perfusion channels is unpredictable and is strongly dependent upon the level of fluid pressure that a clinician applies to inflate the balloon during a procedure. The radially-outward pressure applied by the balloon against an implant or a lesion may also be inconsistent and unpredictable.

The balloon structures taught by U.S. Pat. No. 5,501,667 are challenging to manufacture, may weaken the balloon and unhelpfully offset the guidewire lumen from the central longitudinal axis.

Against this background, the invention resides in a balloon for a perfusion balloon catheter, the balloon comprising: a flexible envelope shaped to define at least two lobes that are mutually spaced angularly about a central longitudinal axis and that project radially, when the envelope is expanded under internal fluid pressure, to define longitudinal perfusion channels outside the envelope between the lobes; and a spine structure within the envelope, wherein the spine structure extends outwardly with respect to the central longitudinal axis to join the envelope at junctions disposed angularly between the lobes in alignment with the perfusion channels. The junctions and the lobes may be equiangularly spaced in circumferential alternation around the central longitudinal axis.

The spine structure also defines a longitudinally-extending spine channel, such as a tube, that surrounds the central longitudinal axis. The tubular spine channel may be circular in cross section or may have a non-circular cross-sectional shape, for example one of variable radius from the central longitudinal axis, with a greater radius in angular alignment with the junctions and a lesser radius in angular alignment with the lobes.

The spine structure may comprise tensile links between the spine channel and the respective junctions, each tensile link lying in a radial plane that intersects the central longitudinal axis. The tensile links may be webs formed integrally with the envelope, and may, like the spine channel, be extruded integrally with the envelope.

The lobes may be blow-moulded portions of the envelope disposed between the webs. In a longitudinally-central portion of the expanded envelope, the lobes may be curved about respective longitudinal axes that are parallel to each other and to the central longitudinal axis. The lobes may, for example, extend around more than 270° of arc about each of their longitudinal axes. The expanded envelope may further comprise transition portions that taper in longitudinally-outward directions from respective ends of the central portion.

At least one of the transition portions may taper down to a longitudinally-protruding tubular stub or neck serving as an inflation tube that is in fluid communication with all of the lobes. The neck may comprise an outer wall that is an integral continuation of the envelope and that may be of substantially circular cross-section.

The neck may be in coaxial alignment with the spine channel. The spine channel may terminate longitudinally inboard of a longitudinally outboard end of the neck, for example within the neck at a position longitudinally outboard of the transition portion that adjoins the neck. Fluid communication with the envelope may conveniently be effected through a gap, such as a radial gap, between the spine channel and the outer wall of the neck. The webs may extend across the gap from the spine channel to the outer wall of the neck, thus helping to keep the gap open.

The inventive concept embraces a perfusion balloon catheter comprising at least one balloon as defined in any preceding claim. The balloon may be mounted on a shaft of the catheter that comprises an inner tube within an outer tube, the outer tube of the shaft being sealed to a proximal end of the balloon and the inner tube of the shaft extending through the balloon along the spine channel and being sealed to a distal end of the balloon.

The outer tube of the shaft may be attached to a neck at the proximal end of the balloon and the inner tube of the shaft may be attached to another neck at the distal end of the balloon. An annulus between the inner and outer tubes of the shaft may be in fluid communication with the envelope. The inner tube of the shaft may open distally and define a longitudinal conduit extending continuously through the balloon.

Correspondingly, the invention extends to a method of making a balloon for a perfusion balloon catheter. The method comprises: placing an elongate element into a mould cavity, the element having an internal profile that defines a spine channel around a central longitudinal axis and that extends outwardly with respect to that axis to join a tubular outer wall of the element at angularly-spaced junctions; and applying internal fluid pressure to the element to expand portions of the outer wall radially within the mould cavity, forming at least two radially-projecting lobes that are mutually spaced angularly about the central longitudinal axis.

The element is oriented in the mould cavity to position the junctions at angular positions between the lobes. In this way, radial expansion of portions of the outer wall in angular alignment with the junctions may be constrained. The portions of the element that form the lobes may be expanded when the element is under longitudinal tension and is heated to a softening temperature.

Preliminarily, end portions of the element may be necked down to a reduced diameter relative to a longitudinally central portion of the element. The end portions may be of substantially circular cross-section whereas the central portion of the element may have a cross-section that is radially enlarged between the junctions. The method may further comprise removing at least part of the internal profile from within the outer wall of the element.

Thus, in embodiments of the invention, the balloon comprises a flexible envelope shaped to define at least two lobes and tensile links located within the envelope, extending radially with respect to the central longitudinal axis to join the envelope at angular positions between the lobes. The tensile links converge on a spine located on the central longitudinal axis, such as a tube that surrounds that axis.

The balloon may further comprise an inflation tube that is in fluid communication with all of the lobes. For example, fluid communication between the inflation tube and the lobes may be effected via at least one transition section of the envelope at an end of the lobes that communicates with the inflation tube through an opening. The opening may be a longitudinal gap disposed within the or each transition section, inboard of an inner end of the inflation tube. For example, a spine may terminate longitudinally at an inboard side of the longitudinal gap. Alternatively, a spine may terminate longitudinally outboard of an inner end of the inflation tube, in which case the opening may be a radial gap between the spine and the inflation tube. In another approach, the opening could be defined by an aperture in a side wall of the inflation tube, for example within the or each transition section.

The or each transition section suitably effects a change in cross-sectional shape of the envelope between the inflation tube and the expanded lobes. For example, the or each transition section may taper down to the inflation tube in a longitudinally-outward direction. Where the balloon comprises a tubular spine, the inflation tube is suitably in coaxial alignment with the spine. The tube of the spine may extend longitudinally to the inflation tube, or short of the inflation tube, or preferably into the inflation tube.

In summary, a catheter balloon of the invention has a single envelope that is shaped to define two or more integral lobes separated by troughs, grooves or other external spaces that provide longitudinal perfusion channels between the lobes. The envelope may conveniently effect fluid communication between a central inflation duct and all of the lobes of the balloon.

A spine structure within the envelope defines a longitudinally-extending spine channel that surrounds a central longitudinal axis. The spine structure also extends outwardly, for example with radially-extending internal webs, to join the envelope at junctions disposed at angular locations between the lobes in alignment with the perfusion channels. The arrangement restrains radially-outward movement of transverse or circumferential webs that form radially-inward bases or apices of the perfusion channels.

Tension in the radial webs balances tension in the walls of the lobes due to expansion. This stabilises the structure of the inflated balloon and maintains its designed shape, particularly the patency of the perfusion channels defined between the lobes and the surrounding vessel wall, even if excessive expansion pressure is applied to the balloon. The radial webs also promote folding of the balloon into a compact collapsed state and predictable and controllable transitions between the expanded and collapsed states and vice versa.

In order that the invention may be more readily understood, reference will now be made, by way of example, to the accompanying drawings in which:

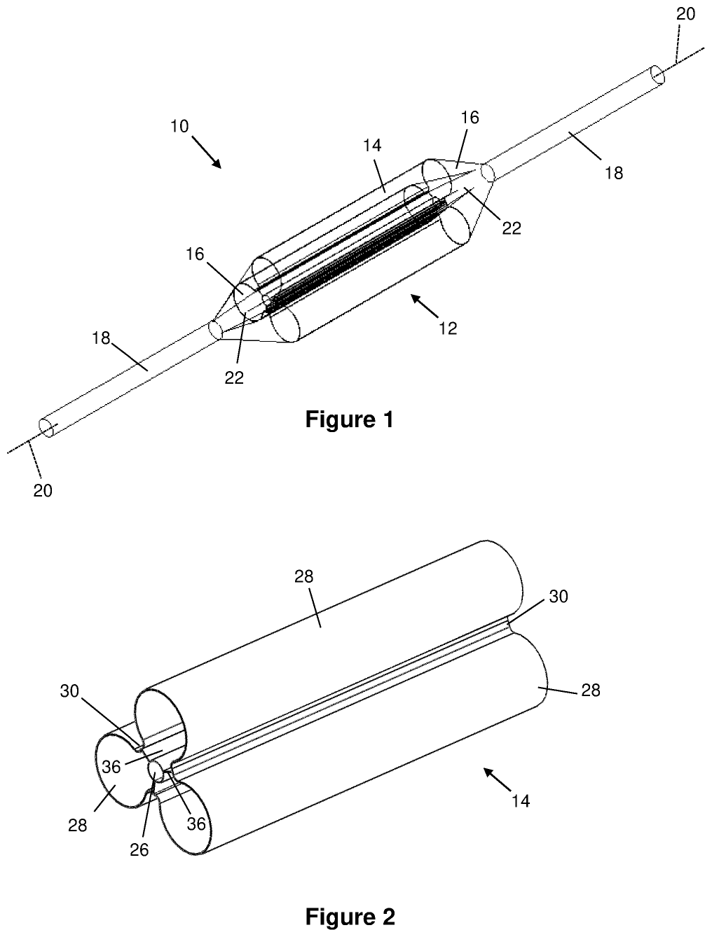

FIG. 1 is a perspective view of a tri-lobal balloon being a first embodiment of the invention, when expanded under internal fluid pressure;

FIG. 2 is a partial perspective view of a central body portion of the balloon of FIG. 1;

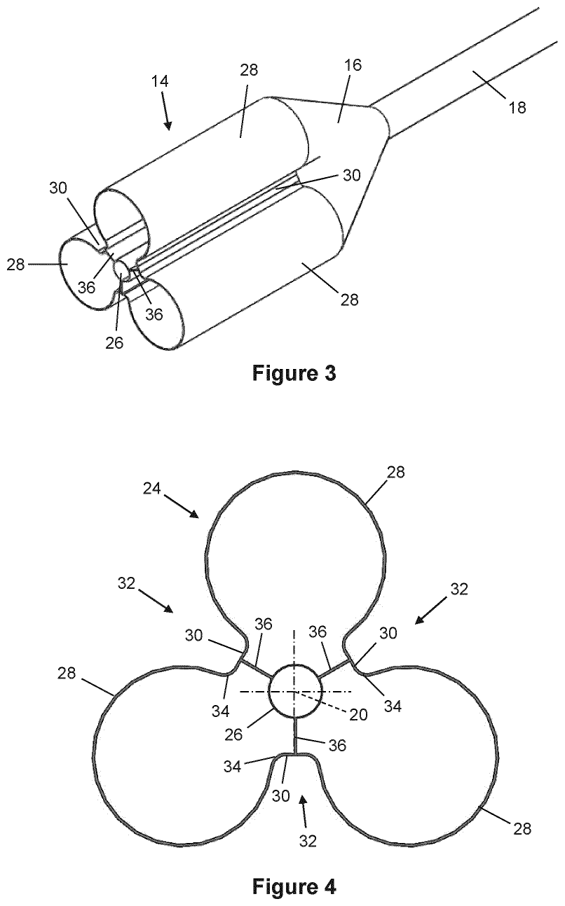

FIG. 3 is an enlarged cut-away view of the balloon of FIG. 1, sectioned through the body portion in a transverse plane that is orthogonal to a central longitudinal axis;

FIG. 4 is a further-enlarged cross-sectional view of the balloon of FIG. 1, taken on the transverse section plane shown in FIG. 3;

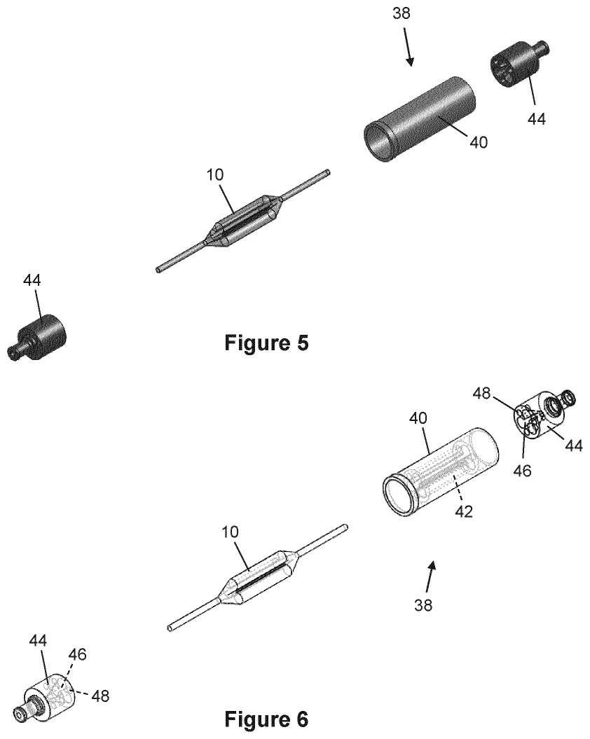

FIG. 5 is an exploded perspective view of the balloon of FIG. 1 and a moulding apparatus for manufacturing the balloon;

FIG. 6 corresponds to FIG. 5 but shows internal features of the moulding apparatus in broken lines;

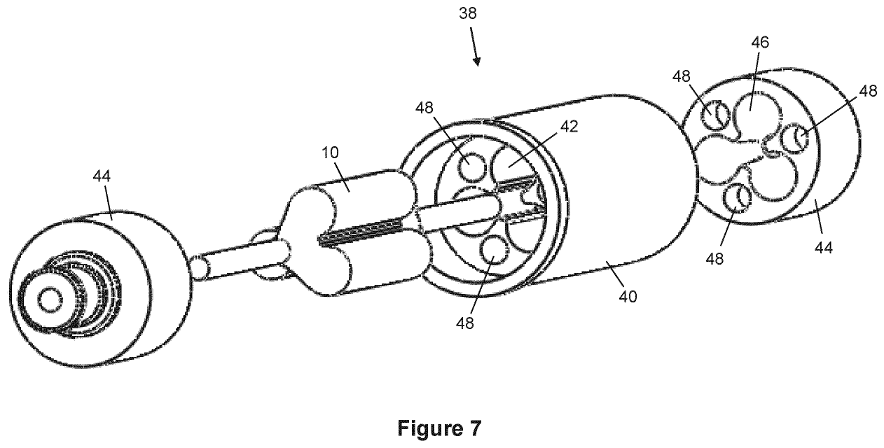

FIG. 7 is an enlarged exploded perspective view of the moulding apparatus of FIGS. 5 and 6;

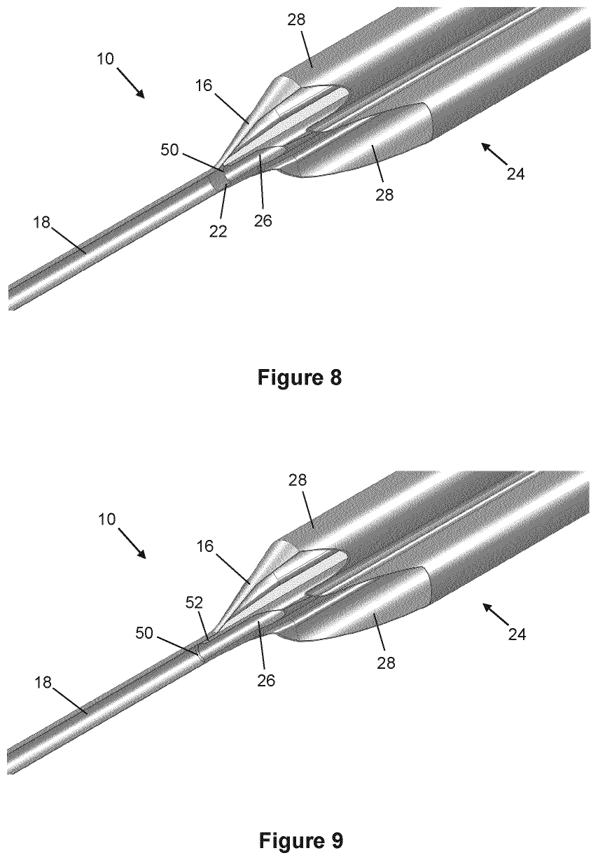

FIG. 8 is a cut-away perspective view of a balloon variant of the first embodiment;

FIG. 9 is a cut-away perspective view of another balloon variant of the first embodiment;

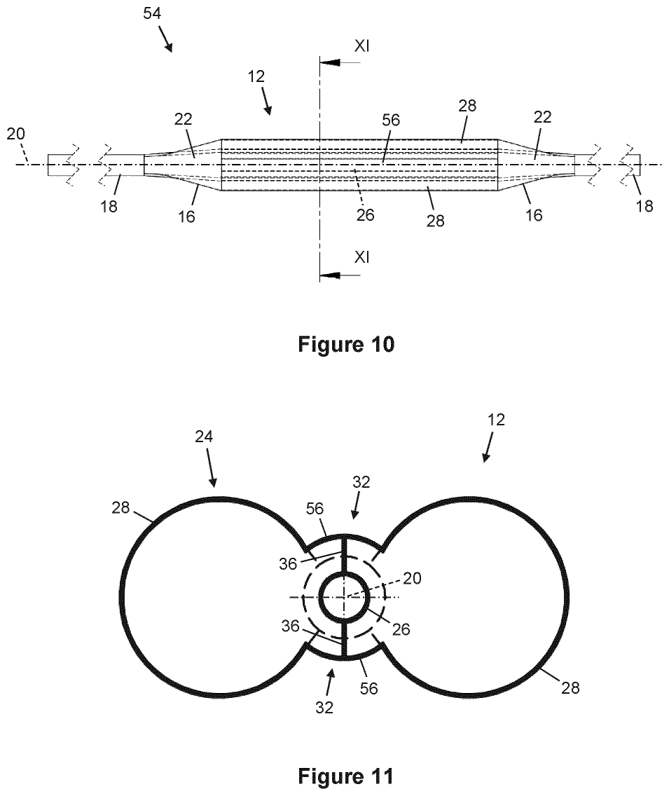

FIG. 10 is a top plan view of a bi-lobal balloon variant of the first embodiment, when inflated;

FIG. 11 is a cross-sectional view of the bi-lobal balloon variant, taken on line XI-XI of FIG. 10;

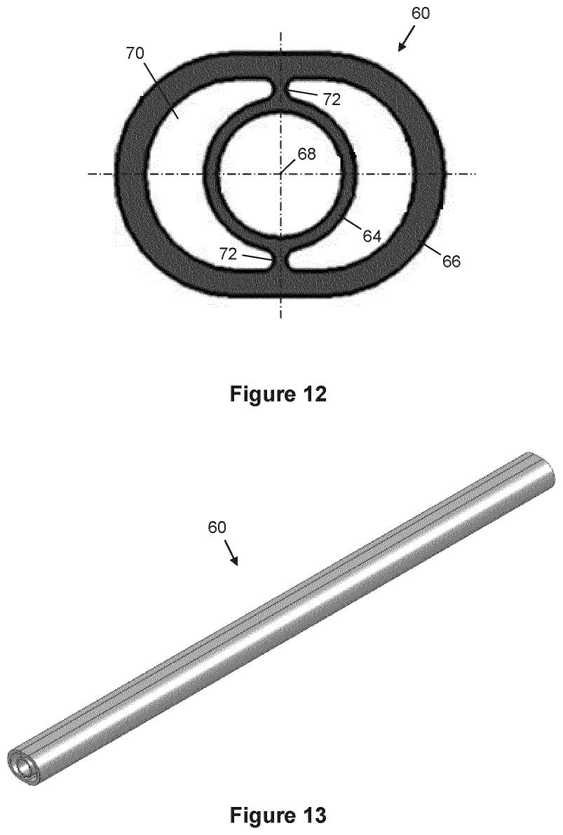

FIG. 12 is a cross-sectional view of an extrusion used in a second embodiment of the invention;

FIG. 13 is a perspective view of the extrusion of FIG. 12 before forming a parison;

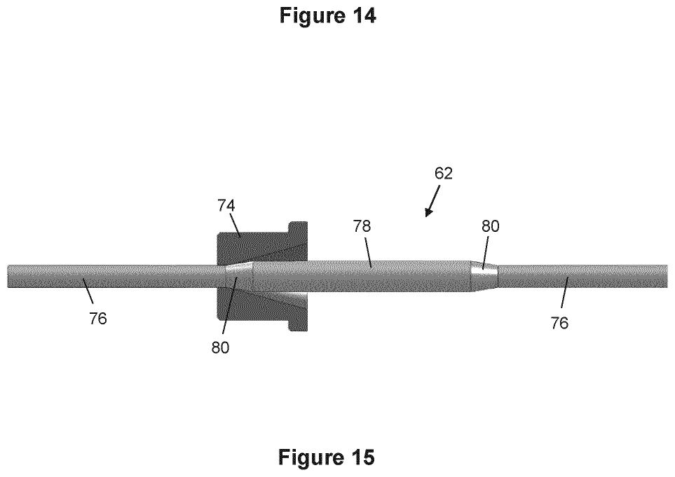

FIG. 14 is a cut-away perspective view of an end portion of the extrusion of FIGS. 12 and 13 being necked down through a heated die to form a parison, the heated die being shown here sectioned in a longitudinal plane;

FIG. 15 is a side view corresponding to FIG. 14;

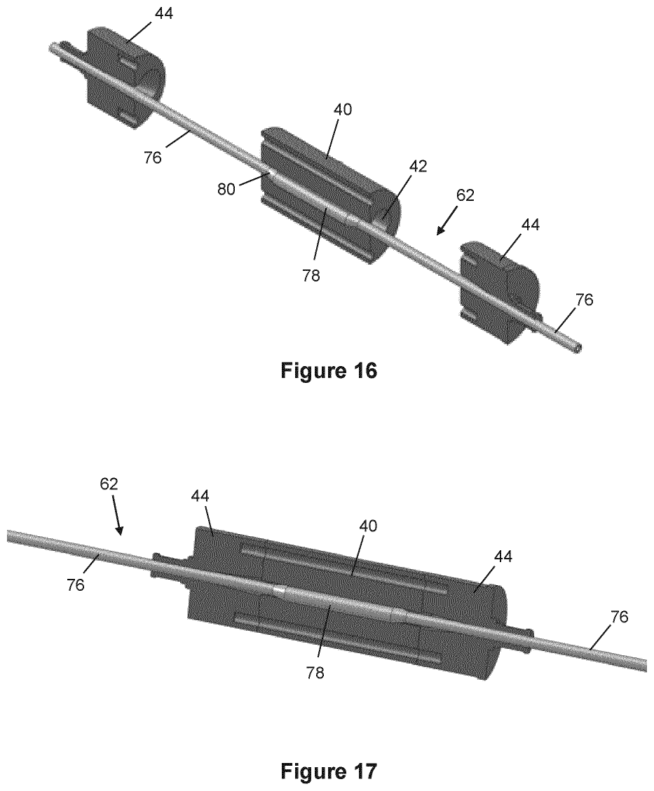

FIG. 16 is a cut-away perspective view of the parison inserted into a mould assembly, the mould assembly being shown here disassembled and sectioned in a longitudinal plane;

FIG. 17 corresponds to FIG. 16 but shows the mould assembly now assembled around the parison;

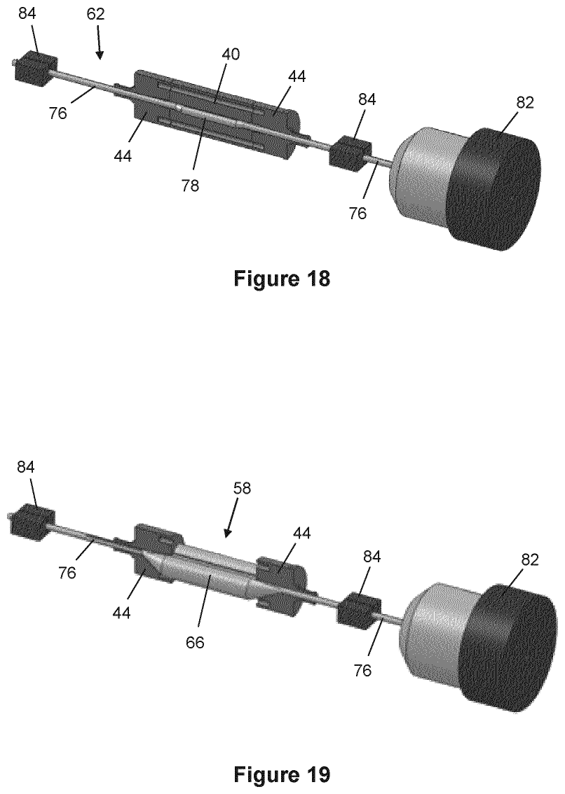

FIG. 18 is a perspective view of the parison in the mould assembly, also showing clamping jaws outside the mould cavity for applying longitudinal tension to the parison and for sealing a distal end of the parison, and a high-pressure gas supply coupled to a proximal end of the parison to inflate the parison within the mould cavity;

FIG. 19 corresponds to FIG. 18 but shows the parison now inflated by the high-pressure gas supply to form a balloon;

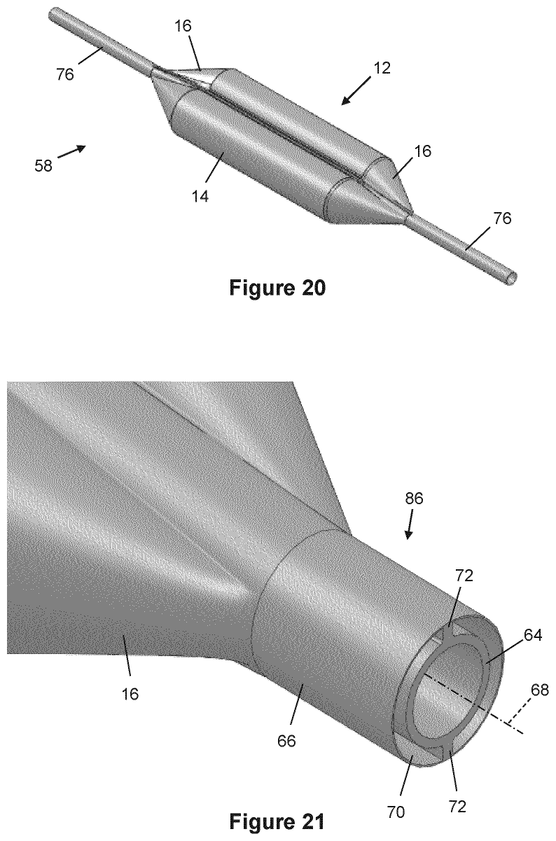

FIG. 20 is a perspective view of the balloon when removed from the mould cavity;

FIG. 21 is an enlarged detail perspective view showing a cut-to-length stub at an end of the balloon;

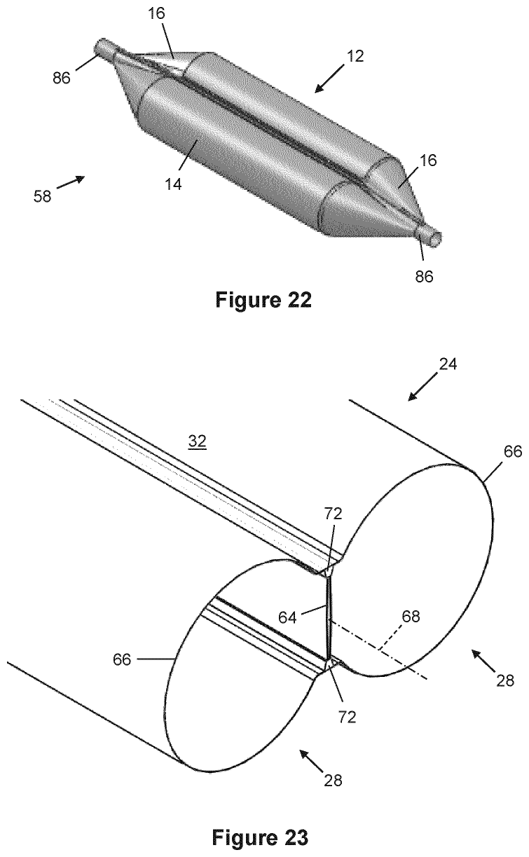

FIG. 22 is a perspective view of the balloon when features within the stub have been cut away;

FIG. 23 is an enlarged detail perspective view of the balloon, sectioned through a body portion of the balloon in a transverse plane that is orthogonal to a central longitudinal axis;

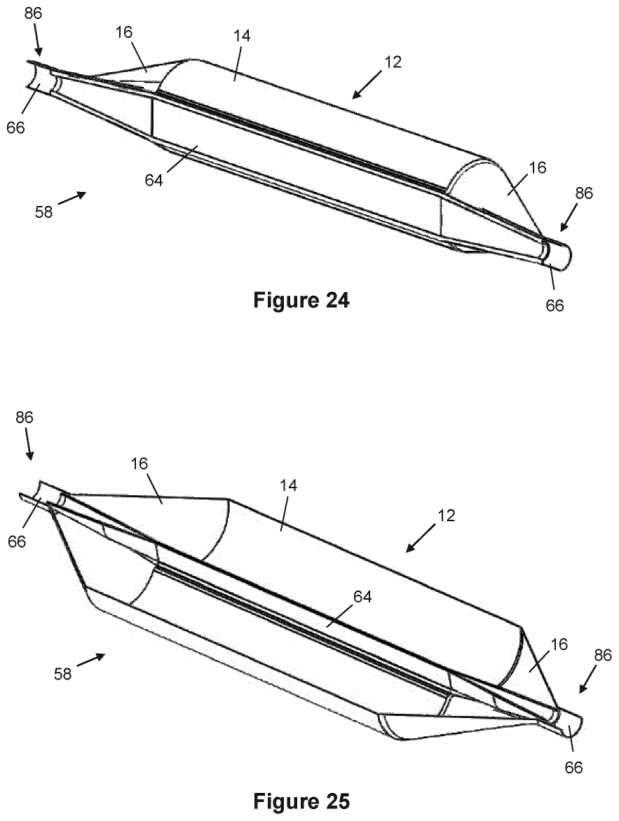

FIG. 24 is a cut-away perspective view of the balloon, sectioned through its narrowest point on a longitudinal plane containing the central longitudinal axis;

FIG. 25 is a cut-away perspective view of the balloon, sectioned through its widest point on a longitudinal plane containing the central longitudinal axis;

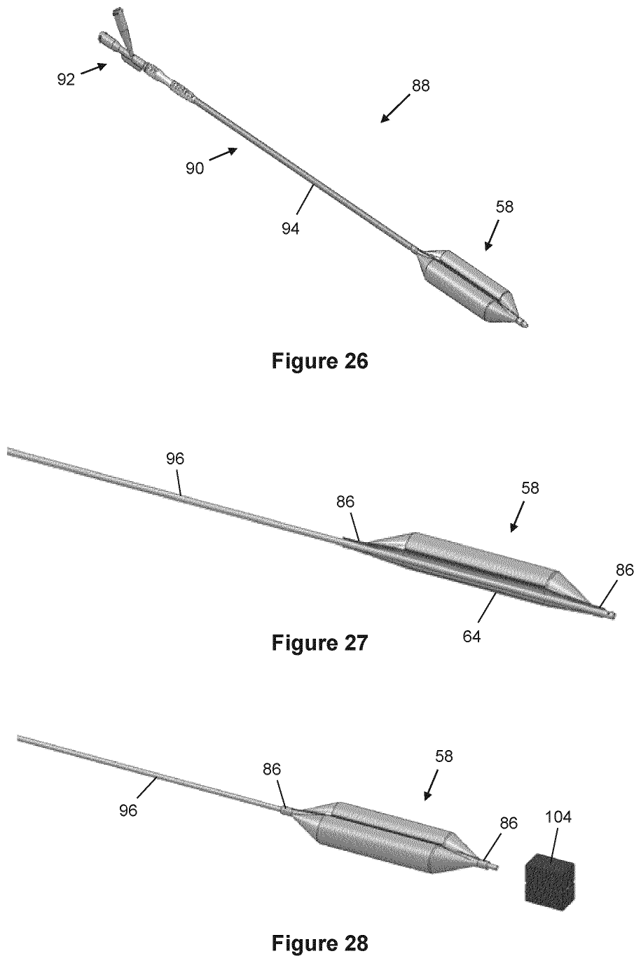

FIG. 26 is a perspective view of a finished assembled catheter with a balloon attached to a tubular shaft of the catheter near a distal end;

FIG. 27 is a cut-away perspective view of a distal end portion of the catheter, showing the balloon sectioned in a longitudinal plane and an inner tube of the catheter shaft extending along the full length of the balloon;

FIG. 28 is a perspective view of the distal end portion of the catheter in conjunction with a heat-shrink device for bonding the balloon to the inner tube of the catheter shaft;

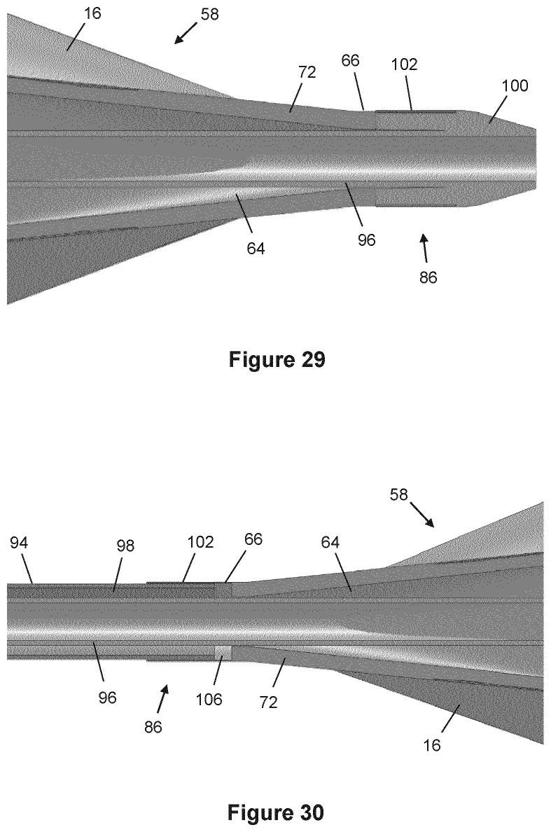

FIG. 29 is an enlarged detail side view in longitudinal section of a joint between the inner tube of the catheter shaft and a distal stub of the balloon; and

FIG. 30 is an enlarged detail side view in longitudinal section of a joint between an outer tube of the catheter shaft and a proximal stub of the balloon.

Referring firstly to FIG. 1 of the drawings, a balloon 10 for a perfusion balloon catheter has a hollow radially-expandable sealed body 12 that comprises an elongate main portion 14, in this example of constant, lobed cross-section, and tapering transition portions 16, one at each end of the main portion 14, that taper away from the main portion 14 in opposite longitudinal directions.

A neck serving as an inflation tube 18 is in fluid communication with the interior of the hollow body 12 and is rotationally symmetrical about a central longitudinal axis 20 that extends along the full length of the balloon 10. The transition portions 16 taper down from the greater diameter of the main portion 14 to the lesser diameter of the substantially narrower inflation tube 18. The transition portions 16 are also fluted to define a gradual transition in cross-sectional shape between the circular cross-section of the inflation tube 18 and the lobed cross-section of the main portion 14.

All of these parts of the balloon 10 are preferably blow-moulded integrally as a single piece, for example by stretch blow moulding a tubular preform under axial tension in a mould or die as will be explained below with reference to FIGS. 5 to 7. However, some of those parts could, in principle, be welded together or otherwise attached to each other.

In this example, the inflation tube 18 is interrupted or divided into two outer parts in mutual alignment, spaced apart by the combined length of the main portion 14 and the transition portions 16 that form the hollow body 12. Those two parts of the inflation tube 18 extend in opposite longitudinal directions away from the outer ends of the tapering portions 16. Also, in this example, the two parts of the inflation tube 18 are joined at their inner ends around their circumference to the transition portions 16 but are spaced longitudinally from the main portion 14 of the body 12.

By virtue of the longitudinal gaps 22 between the two parts of the inflation tube 18 and the main portion 14 of the body 12, the inflation tube 18 communicates with the hollow interior of the main portion 14 via the transition portions 16. This provides for inflation and radial expansion of the body 12 by the application of internal fluid pressure to the inflation tube by a gas such as air or a liquid such as water. The transition portions 16 therefore serve as manifolds that channel pressurising fluid from the inflation tube 18 into the lobes of the main portion 14.

Additionally, the inflation tube 18 defines a lumen that, if required, can also accommodate an elongate guide element (not shown) that extends longitudinally through the balloon 10, such as a wire for over-the-wire access. In this example, the single lumen of the inflation tube 18 serves both purposes but variants of the invention could have a central tube with two or more parallel lumens for respectively different purposes such as inflation and guidewire access.

FIG. 2 shows the main portion 14 of the body 12 in isolation whereas FIG. 3 shows part of the main portion 14, the inflation tube 18 and one of the transition portions 16 that extends between the main portion 14 and the inflation tube 18. FIG. 4 shows the cross-sectional profile of the main portion 14 in isolation, which will now be described.

The body 12 comprises a continuous, flexible envelope 24 of fluid-impermeable polymer material. The envelope 24 surrounds a spine structure comprising a central tubular spine 26 that is centred on the longitudinal axis 20 also shown in FIG. 1 and so is in coaxial alignment with the inflation tube 18. The spine 26 could, however, become elongated or flattened to some extent when the envelope 24 of the balloon 10 is inflated.

In this example, the spine 26 extends longitudinally for the full length of the main portion 14 of the body 12 but no further, and so terminates short of the two parts of the inflation tube 18 to define inner ends of the gaps 22. In other examples to be described later with reference to FIGS. 8 and 9, the spine 26 extends into the transition sections 16 to terminate at a longitudinal position that is close to or even beyond the outer ends of the transition sections 16, at the interface where the envelope 24 splays away from the inflation tube 18.

By virtue of successive inflections of curvature, the envelope 24 undulates in radially-inward and radially-outward directions to define male and female formations that alternate circumferentially around the central longitudinal axis 20. Specifically, the envelope 24 has radially-protruding lobes 28 that alternate circumferentially around the axis 20 with radially-depressed troughs or grooves 30. In this tri-lobal variant, the lobes 28 and hence the grooves 30 are equi-angularly spaced at 120° intervals around the central longitudinal axis 20. The grooves 30 define perfusion channels 32 in the longitudinally-extending spaces between the lobes 28 outside the envelope 24.

The lobes 28 are each of part-elliptical, preferably part-circular, cross-section as shown, with curvature centred on respective longitudinal axes that are parallel to each other and to the central longitudinal axis 20. The part of the envelope 24 defining the wall of each lobe 28 extends around more than 270° of arc; approximately 300° of arc in this example.

Parallel radiused edges 34 of the base of each groove 30 blend smoothly into the arcs of the lobes 28 to each side of the groove 30. The arcs of those lobes 28 then splay apart from each other moving radially outwardly from the base of the groove 30.

In accordance with the invention, the spine structure further comprises radially-extending tensile links exemplified here by integral webs 36 join to the radially-inward side of the base of each groove 30. Thus, the webs 36 join the envelope 24 at respective junctions aligned with each groove 30. In this example, the webs 36 join the base of each groove 30 to the central spine 26 to couple the base of each groove 30 to the spine 26. The webs 36 extend continuously along the full length of the main portion 14 of the body 12, thus joining the full length of the spine 26 to the full length of the base of each groove 30.

Again, in this tri-lobal variant, the webs 36 are equi-angularly spaced at 120° intervals around the spine 26 and the central longitudinal axis 20, in planes that would angularly bisect the respective perfusion channels 32 if extended outwardly beyond the base of the grooves 30. Each web 36 therefore intersects the base of a groove 30 centrally, hence bisecting the base of the groove 30 longitudinally.

Beneficially, the webs 36 prevent the base of each groove 30 being pulled radially outwardly away from the central longitudinal axis 20 by virtue of internal fluid pressure in the lobes 28. In this respect, it will be noted that fluid pressure in the lobes 28 will create tension and hoop stress in the parts of the envelope 24 that define the lobes 28. Without the inward reaction of tension in the constraining webs 36, the base of each groove 30 would be pulled outwardly to an uncontrolled and unpredictable extent, eventually reaching an equilibrium radial position that is determined only by the fluid pressure within the balloon 10.

Without the webs 36, the structure of the inflated balloon 10 would be unstable and of unpredictable cross-sectional shape. In extremis, excessive fluid pressure within the balloon 10 could push the base of the grooves 30 so far outwardly that the cross-sectional area of the perfusion channel 32 becomes restricted, potentially restricting blood flow to a dangerous extent.

In the cross-section illustrated, the base of each groove 30 is nominally flat. In practice, when the body 12 of the balloon 10 is fully inflated, the base of each groove 30 may adopt a shallow V-section shape under the opposing tensions of the central web 26 acting between, and against, the parts of the envelope 24 that define the adjoining lobes 28.

Whilst the continuous webs 36 and spine 26 isolate the lobes 28 from each other in cross-section, the tapering portions 16 of the body 12 effect fluid communication between the inflation tube 18 and all of the lobes 28. Conveniently, therefore, fluid introduced via a single lumen of the inflation tube 18 will simultaneously and equally inflate all of the lobes 28 with balanced fluid pressure.

Being integrally moulded with the rest of the main portion 14 of the body 12, the webs 36 are created by a balloon forming process that will now be described with reference to the moulding apparatus 38 shown in FIGS. 5 to 7 of the drawings.

A pre-formed tubular extrusion is placed into a mould body 40 of the moulding apparatus 38. The shape of the internal cavity 42 of the mould body 40 determines, and hence corresponds to, the shape of the main portion 14 of the balloon body 12. The extrusion extends along and through the full length of the mould body 40 and overlaps beyond the ends of the mould body 40. The extrusion has an outer tubular wall that corresponds to the envelope 24 of the balloon, extruded integrally with an inner profile that corresponds to the spine 26 and the webs 36.

Cone sleeves 44 are fitted to opposite ends of the mould body 40 to close the mould cavity around the extrusion. The internal cavities 46 of the cone sleeves 44 correspond in shape to the transition portions of the balloon body 12. The opposed ends of the extrusion protrude longitudinally beyond the cone sleeves 44.

Dowel locator holes 48 are used to align the cone sleeves 44 with the mould body 40 by virtue of dowel rods or pins that run through the holes 48 from one cone sleeve 44 through the mould body 40 to the corresponding holes 48 of the opposite cone sleeve 44. In this way, all of the components of the moulding apparatus 38 are held together in correct alignment.

The moulding apparatus 38 and the extrusion within it are heated while high internal pressure is applied to the extrusion to act against the outer tubular wall of the extrusion. Then, after a suitable period of heating to soften its polymer material, the extrusion is stretched in the mould cavity 42, 46 by pulling on the ends of the extrusion that protrude beyond the cone sleeves 44. As the extrusion stretches and lengthens under this longitudinal tension, its walls thin out until the outer tubular wall blows out under the elevated internal pressure into the shape of the mould cavity 42, 46 defined within the assembly of the mould body 40 and the cone sleeves 44.

The whole moulding apparatus 38 is then cooled while maintaining internal pressure within the blow-moulded component until the polymer of that component reaches a setting temperature, thus locking in the desired balloon shape imparted by the internal profile of the mould. The internal pressure in the blow-moulded extrusion can then be relieved to ambient pressure before the balloon 10 is removed from the mould body 40.

Finally, the web material within the tapering transition portions 16 of the balloon body 12 is removed by: manual cutting, for example using a smooth-bore hypotube, a thin blade or a mandrel; high-speed cutting with a sharp cutting tool such as an end mill or drill; laser cutting; or water jet cutting. More generally, the inner profile of the extrusion can either be pierced or cut away to a desired longitudinal extent from the outer end of at least one of the inflation tubes 18. This completes the balloon forming process.

The inner profile of the extrusion is removed to some extent, at least in a proximal section of the inflation tube 18, so that the inflation tube 18 can accommodate an outer lumen or extrusion of a catheter to be attached to the balloon 10. There is no corresponding need to remove the inner profile of the extrusion from the distal section of the inflation tube 18, although its removal may still be beneficial to reduce the material in that section and so to ease navigation of the catheter.

Whilst it is advantageous to remove or cut the inner profile of the extrusion in some circumstances, this is not essential in all circumstances. In this respect, it will be apparent that even if the inner profile within the inflation tubes 18 is not cut away, the envelope could be inflated by fluid pressure applied to the annular gap between the inner profile and the outer wall of the inflation tube 18.

As mentioned above, FIGS. 8 and 9 show that the spine 26 can extend longitudinally beyond the body 12 into the transition sections 16 to terminate close to or even beyond the interface where the envelope 24 splays away from the inflation tube 18. In this respect, it has been found that the inner profile of the extrusion is best terminated either just inside the transition section 16 or just inside the inflation tube 18, say within 3 mm to either side of the outer end of the transition section 16. If the inner profile of the extrusion is terminated too far into the envelope 24, so that the longitudinal gap 22 is too big, then the web of the envelope 24 would be more prone to tearing when the balloon 10 is pressurised. This would reduce the rated burst pressure of the balloon 10.

FIG. 8 shows the cut outer end 50 of the spine 26 defined by the inner profile of the extrusion located slightly inboard of the inner end of the inflation tube 18, and hence just outside the inflation tube 18. Consequently, a small longitudinal gap 22 remains between the inner end of the inflation tube 18 and the outer end of the spine 26. The inflation tube 18 communicates with all of the lobes 28 of the envelope 24 through that gap 22.

In FIG. 9, conversely, the cut outer end 50 of the spine 26 is located slightly outboard of the inner end of the inflation tube 18, hence just within the inflation tube 18. Consequently, the inflation tube 18 communicates with all of the lobes 28 through a small annular radial gap 52 that remains between the outer end of the spine 26 and the inner end of the inflation tube 18.

Turning to FIGS. 10 and 11, these figures show a balloon 54 being a bi-lobal variant of the invention, in which like numerals are used for like features. In this example, the envelope 24 of the body 12 again surrounds a spine structure comprising a central tubular spine 26 that is centred on a longitudinal axis 20 in coaxial alignment with the inflation tube 18. Again, longitudinal gaps 22 are left between the ends of the spine 26 and the two parts of the inflation tube 18. The gaps establish fluid communication between the inflation tube 18 and the radially-protruding lobes 28 of the body via the tapering transition portions 16.

In this bi-lobal variant, the lobes 28 and hence the perfusion channels 32 defined by the spaces between the lobes 28 are equi-angularly spaced at 180° intervals around the central longitudinal axis 20. Again, the lobes 28 are each of part-elliptical, preferably part-circular, cross-section and the part of the envelope 24 defining the wall of each lobe 28 extends around more than 270° of arc; approximately 300° of arc in this example.

In this instance, the lobes 28 are joined by circumferential webs 56 that equate in function to the bases of the grooves 30 of the tri-lobe variant. Radially-extending integral webs 36 of the spine structure serving as tensile links join the radially-inward sides of the circumferential webs 56 to the central spine 26.

In this bi-lobal variant, the radial webs 36 are equi-angularly spaced at 180° intervals around the spine 26 and the central longitudinal axis 20, in planes that would angularly bisect the respective perfusion channels 32 if extended outwardly beyond the circumferential webs 56. The radial webs 36 therefore intersect the circumferential webs 56 centrally, hence bisecting the circumferential webs 56 longitudinally.

Many other variations of the preceding arrangements are possible within the inventive concept. For example, the inflation tube 18 could, in principle, extend from only one end of the inflatable body 12.

The spine 26 is preferably tubular to accommodate a guidewire that also extends along the inflation tube. However, in principle, the spine 26 could instead be a solid elongate member such as a rod, or the webs 36 or other links could simply converge and adjoin directly on the central longitudinal axis 20.

Fluid communication between the inflation tube 18 and the interior of the body 12 could be effected through an opening other than a gap between the inflation tube 18 and the main portion 14 of the body 12. For example, the inflation tube 18 could extend to, and possibly longitudinally through, the main portion 14 and at least one aperture could be provided or created in a side wall of the inflation tube 18 in line with a transition portion 16 of the body 12.

In principle, it would be possible for the spine 26 to be longer than the main portion 14, or to extend longitudinally beyond the main portion 14 at one or both ends. For example, the spine 26 could extend to the ends of the transition portions 16 to join with the opposed parts of the inflation tube 18. Thus, the spine 26 and the inflation tube 18 could be continuous through the full length of the balloon 10. Put another way, the inflation tube 18 could also serve as the spine 26 or the spine 26 could also serve as the inflation tube 18.

It would also be possible for the spine 26 to be shorter than the length of the main portion 14 so as to be recessed into one or both ends of the main portion 14.

Fluid communication between the inflation tube 18 and the interior of the body 12 could be effected through an opening at only one end of the body 12.

Moving on now to FIGS. 12 to 30, these drawings show a further embodiment of the invention that is again exemplified by a bi-lobal perfusion balloon 58. It will be clear to the skilled reader that similar principles of construction and manufacture could be applied to a tri-lobal variant.

FIGS. 12 and 13 show an extrusion 60 that is used to form a parison 62 for moulding the balloon, as shown in FIGS. 14 and 15. The extrusion 60 is of thermoplastic polymer material such as Nylon 12 or Pebax, this being an elastomer offered by Arkema Group (registered trade marks acknowledged).

As best seen in FIG. 12, the extrusion 60 comprises an inner tubular wall 64 of circular cross-section and an outer tubular wall 66 of stadium cross-section, both centred on a central longitudinal axis 68. The outer wall 66 of the extrusion 60 is thicker than the inner wall 64 to provide a sufficient volume of material for stretch blow moulding the parison 62 to form the balloon, as will be explained.

The outer wall 66 is spaced radially from the inner wall 64 to define an annular gap 70 between them. Integral webs 72 equi-angularly spaced at 180° intervals around the inner wall 64 extend radially in longitudinal planes across the gap 70 from the inner wall 64 to the outer wall 66. The webs 72 join the outer wall 66 at central positions on the flat parallel sides of its stadium cross section, hence at the narrowest point of the cross-section of the outer wall 66.

The extrusion 60 is cut to a length appropriate for the balloon 58, as shown in FIG. 13, before being formed into a parison 62 as shown in FIGS. 14 and 15. In those drawings, the parison 62 is shown being formed by applying longitudinal tension to the extrusion 60 and pulling each opposed end portion of the extrusion 60 through a heated die 74 with a generally conical, tapering cavity whose downstream orifice is of circular cross section. The die 74 may, for example, be heated to a temperature of 120° C. to 140° C.

This necking process reshapes the opposed end portions 76 of the extrusion 60, bringing them to the circular cross-sectional shape that is appropriate for the corresponding interface formations at the ends of the finished balloon 58. The necking process also narrows down the end portions 76, bringing them down to the outer diameter that is required for them to fit within cone sleeves 44 at the ends of a mould assembly shown in FIGS. 16 and 17.

In a wider central portion 78 between the end portions 76, the parison 62 retains the size and aforementioned cross-sectional shape of the original extrusion 60. Outwardly-tapering transitions 80 between the end portions 76 and the central portion 78 reflect the tapered shape of the cavity within the die 74.

Next, with reference to FIGS. 16 and 17, the parison 62 is inserted into a mould assembly comprising a mould body 40 and distal and proximal cone sleeves 44 that are akin to the corresponding parts of the moulding apparatus 38 described above with reference to FIGS. 5 to 7, albeit shaped to suit this bi-lobal variant. These parts of the mould assembly are typically machined from a metal such as brass.

The central portion 78 of the parison 62 is received in a cavity in the mould body 40, the transitions 80 between the end portions 76 and the central portion 78 are received in cavities in the respective cone sleeves 44, and the end portions 76 protrude from the mould assembly through the cone sleeves 44. The cavities of the mould body 40 and the cone sleeves 44 together define a mould cavity 42 of constant cross-section terminating in tapering ends.

The flattened cross-section of the central portion 78 of the parison 62, corresponding to the original extrusion 60, requires the parison 62 to be in a particular angular orientation to fit into the mould cavity 42. Specifically, the rounded sides of the outer wall 66 face into concave recesses of the mould cavity 42 that provide clearance for those parts of the outer wall 66 to expand outwardly. Conversely, expansion of the outer wall 66 is restrained in orthogonal directions because the mould cavity 42 fits closely around the central portion of the parison 62 in alignment with the webs 72 that join the outer wall 66 to the inner wall 64.

FIGS. 18 and 19 show the proximal end of the parison 62 now in fluid communication with a high-pressure supply of gas 82, such as nitrogen or air, and the distal end of the parison 62 sealed by clamping it and/or heating and melt-sealing it. For this purpose, a clamp 84 is shown around the distal end portion 76 of the parison 62. Another clamp 84 is shown around the proximal end portion 76 of the parison 62. The clamps 84 may, for example, comprise pneumatic jaws. The clamps 84 can be driven apart in opposite longitudinal directions, for example by electric motors, to put the parison 62 under longitudinal tension.

As described above with reference to FIGS. 5 to 7, the mould assembly and the parison 62 within it are placed into a heater block on a stretch blow moulding machine and heated while high internal pressure is applied to the parison 62 to act against the outer wall 66 of the parison 62. The parison 62 is longitudinally stretched in the mould cavity 42 while under internal pressure by pulling on the ends of the protruding sleeves via the clamps 84. As the parison 62 reaches a softening temperature and is stretched under internal pressure, its walls 64, 66 thin out until the outer wall 66 blows out under the elevated internal pressure, hence forming the shape of the balloon 58 in the mould cavity 42. This is shown in FIG. 19, in which the mould body 40 has been removed for clarity.

The balloon 58 is heat-set in the mould cavity 42 for a predetermined length of time and then cooled via a cooling circuit within the heater block to maintain the desired shape imparted by the mould cavity 42. For example, once the balloon 58 reaches a desired heat-set temperature of typically between 120° C. and 150° C., it may be allowed to heat-set for approximately 30 to 60 seconds. Once this period is complete, the mould assembly may be cooled down to a temperature of about 20° C. to 22° C.

The mould assembly is then opened, and the resulting balloon 58, as shown in FIG. 20, is removed for further processing. Firstly, the balloon 58 undergoes inspection for visual and dimensional acceptance criteria. Then, as shown in FIG. 21, the parts of the balloon 58 corresponding to the end portions 76 of the parison 62 are cut back, leaving short necks in the form of spigots or stubs 86 protruding longitudinally by about 5 mm to 10 mm from the tapering transition portions 16 of the balloon body 12. The stubs 86 are of circular cross section comprising inner and outer walls 64, 66 joined by radial webs 72 and spaced apart by an annular gap 70, in concentric relation about a central longitudinal axis 68. All of these features have counterparts in the corresponding features of the original extrusion 60, and so have the same numbering here and in the drawings.

Once the stubs 86 are cut to length, the inner wall 64 and the webs 72 are removed from within the outer wall 66 of each stub 86, resulting in the final balloon arrangement shown in FIG. 22. The inner wall 64 may, for example, be cut through circumferentially with a rotary tool having a circular array of cutting teeth on a rotary head that can fit within the inner wall 64. Then, after cutting through the webs 72, for example with a sharp-edged circular-section hollow tube, tweezers may be used to remove the cut section of the inner wall 64 from within the outer wall 66. Alternatively, a rotary head with a circular array of cutting teeth that can fit within the outer wall 66 could be used to mill away both the inner wall 64 and the webs 72 simultaneously.

In principle, a length of the inner wall 64 and the webs 72 equal to or greater than the length of the stubs 86 could be removed, thus cutting the inner wall 64 and the webs 72 back to, or into, the tapered transition portions 16 of the balloon body 12. However, it is preferred that a length of the inner wall 64 and the webs 74 slightly less than the length of the stubs 86 is removed so that the inner wall 64, at least, extends slightly into the stubs 86, to a position outboard of the junction between the transition portions 16 and the stubs 86. For example, the inner wall 64 may extend up to 1 mm outboard of the interface where a stub 86 joins a transition portion 16. This is preferred because the alternative of removing the inner wall 64 into the transition portions 16 could result in weakness when the balloon 58 is under pressure, potentially causing the envelope 24 to tear and fail during deployment.

As best appreciated in the transverse cross-section of FIG. 23, the outer wall 66 of the extrusion 60, and particularly the rounded ends of its cross-section, define a flexible envelope 24 comprising two lobes 28 that are equi-angularly spaced at 180° intervals about the central longitudinal axis 68. When expanded under internal fluid pressure, the lobes 28 project radially to define longitudinal perfusion channels 32 outside the envelope 24 between the expanded lobes 28.

The internal profile of the extrusion 60 comprises the inner wall 60 and the webs 72 that together define a spine structure. The inner wall 64 defines a tubular spine from which the webs 72 extend radially with respect to the central longitudinal axis 68 to join the envelope 24 at angular positions between the lobes 28 in alignment with the perfusion channels 32. Thus, the webs 72 join the inner wall 64 to the outer wall 66 at respective junctions. This is similar to the cross-section of the preceding bi-lobal variant shown in FIG. 11, except that here the tubular spine defined by the inner wall 64 is flattened and thinned into a generally elliptical cross-section that extends radially in a direction aligned with the webs 72, close to the central longitudinal axis 68.

To maintain the shape of the balloon 58 after cutting the stubs 86 to length and removing the inner wall 64 and the webs 72 from within the outer wall 66 of the stubs 86, the balloon 58 is inflated under an internal pressure of about one bar above ambient and annealed in an oven at say 60° C. for fifteen minutes. Once annealed, the balloon 58 is removed from the oven, undergoes a final visual inspection, and is then packed ready for shipment.

Referring to FIGS. 24 and 25, these longitudinal sections show the spine defined by the flattened, thinned inner wall 64 within the balloon body 12. As the stubs 86 do not expand during the blowing process, the inner wall 64 remains approximately circular within the stubs 86. Thus, as they extend through the transition portions 16 of the balloon body 12, the halves of the inner wall 64 on opposite sides of the webs 72 transform from nearly flat within the main portion 14 of the balloon body 12 to semi-circular within the stubs 86. FIGS. 24 and 25 also show the extent to which the inner wall 64 is cut back from the end of the outer wall 66 of each stub 86.

FIGS. 26 to 30 put the balloon 58 into its context of use as part of a balloon catheter. In this respect, FIG. 26 shows a catheter assembly 88 comprising a balloon 58 of the invention mounted on a distal end portion of a catheter shaft 90. A manifold 92 is attached to the proximal end of the catheter shaft 90 via a conventional strain relief feature.

As best appreciated in FIG. 30, the catheter shaft 90 comprises outer and inner tubes 94, 96 in concentric relation, defining an annular channel 98 between them through which fluid pressure can be applied to inflate the balloon 58. Thus, the outer tube 94 serves as an inflation tube. The manifold 92 is bonded to the outer and inner tubes 94, 96 of the catheter shaft 90 in a manner that maintains fluid communication between the balloon 58 and the manifold 92 through the annular channel 98.

FIGS. 27 to 29 do not show the outer tube 94 but instead show that the inner tube 96 of the catheter shaft 90 extends through the full length of the balloon 58, though and along the channel defined by the inner wall 64. The inner tube 96 terminates distally beyond, and is sealed to, the distal end stub 86 of the balloon 58. The inner tube 96 may, for example, extend 10 mm to 15 mm beyond the distal end of the distal end stub 86. FIG. 29 shows a taper formation applied to the projecting distal tip 100 of the inner tube 96, for example by reshaping the distal tip 100 with heat, for ease of entry and navigation of the balloon 58 and the catheter shaft 90 in use.

FIG. 29 also shows heat-shrink tubing 102 placed around the distal tip 100 and the distal stub 86 of the balloon 58, hence effecting sealing attachment between the inner tube 96 and the balloon 58. Conveniently, heat that shrinks the heat-shrink tubing 102 may also reshape the distal tip 100. For this purpose, FIG. 28 shows a laser welder or hot jaw bonder 104 that can embrace the distal tip 102 and the distal stub 86 of the balloon 58.

FIG. 30 shows that the outer tube 94 of the catheter shaft 90 terminates distally within, and is sealed to, the proximal stub 86 of the balloon 58. FIG. 30 also shows heat-shrink tubing 102 placed around the junction between the proximal stub 86 and the distal end of the outer tube 94, hence effecting sealing attachment between the outer tube 94 and the balloon 58. The outer tube 94 may be pushed distally up to the cut inner wall 64 and web 72 section of the balloon 58 but preferably, as shown, terminates longitudinally outboard of that point. In this respect, an annular radial gap 106 between the proximal end of the inner wall 64 and the distal end of the outer tube 94 ensures that inflation of the balloon 58 is not impeded.

By extending along the channel defined by the inner wall 64 of the balloon 58, the inner tube 96 defines a continuous lumen that allows an elongate element such as a guidewire or a therapeutic or surgical device to extend along the catheter shaft 90 and to emerge distally from the distal end of the balloon 58.

1. A balloon for a perfusion balloon catheter, the balloon comprising:
a flexible envelope defining at least two lobes that are disposed mutually spaced angularly about a central longitudinal axis and that project radially, when the envelope is expanded under internal fluid pressure, to define a plurality of longitudinal perfusion channels outside the envelope between the at least two lobes; and
a spine structure arranged within the envelope;
wherein the spine structure extends outwardly with respect to the central longitudinal axis to join the envelope at a plurality of junctions disposed angularly between the at least two lobes in alignment with the plurality of perfusion channels; and
wherein the spine structure defines a longitudinally-extending spine channel that surrounds the central longitudinal axis.

2. The balloon of claim 1, wherein:
the spine structure includes a plurality of tensile links disposed between the spine channel and the respective junctions; and
each tensile link of the plurality of tensile links lies in a radial plane that intersects the central longitudinal axis.

3. The balloon of claim 1, wherein the spine channel is defined by a tube that surrounds the central longitudinal axis.

4. The balloon of claim 3, wherein:
the tube has a cross-sectional shape of variable radius from the central longitudinal axis; and
the cross-sectional shape of the tube has a greater radius in angular alignment with the plurality of junctions and a lesser radius in angular alignment with the at least two lobes.

5. The balloon of claim 2, wherein the plurality of tensile links are a plurality of webs formed integrally with the envelope.

6. The balloon of claim 5, wherein;
the plurality of webs are extruded integrally with the envelope; and
the at least two lobes are blow-moulded portions of the envelope disposed between the plurality of webs.

7. The balloon of claim 6, wherein the spine channel is extruded integrally with the plurality of webs and the envelope.

8. The balloon of claim 1, wherein the plurality of junctions and the at least two lobes are arranged equiangularly spaced in circumferential alternation around the central longitudinal axis.

9. The balloon of claim 1, wherein, in a longitudinally-central portion of the expanded envelope, the at least two lobes are curved about respective longitudinal axes that are parallel to each other and to the central longitudinal axis.

10. The balloon of claim 9, wherein the at least two lobes each extend around more than 270° of arc about their respective longitudinal axis.

11. The balloon of claim 9, wherein the expanded envelope includes a plurality of transition portions that taper in longitudinally-outward directions from respective ends of the longitudinally-central portion of the expanded envelope.

12. The balloon of claim 11, wherein;
at least one transition portion of the plurality of transition portions tapers down to a longitudinally-extending tubular neck; and
the neck includes an outer wall that is an integral continuation of the envelope.

13. The balloon of claim 12, wherein the outer wall of the neck has a substantially circular cross-section.

14. The balloon of claim 12, wherein the spine channel terminates longitudinally inboard of a longitudinally outboard end of the neck.

15. The balloon of claim 14, wherein the spine channel terminates within the neck, longitudinally outboard of the at least one transition portion adjoining the neck.

16. The balloon of claim 12, wherein the neck is an inflation tube that is in fluid communication with all of the at least two lobes.

17. The balloon of claim 12, wherein fluid communication with the envelope is provided via a gap defined between the spine channel and the outer wall of the neck.

18. The balloon of claim 17, wherein the gap is a radial gap defined between the spine channel and the outer wall of the neck.

19. The balloon of claim 18, wherein;
the spine structure includes a plurality of tensile links disposed between the spine channel and the respective junctions;
each tensile link of the plurality of tensile links lies in a radial plane that intersects the central longitudinal axis;
the plurality of tensile links are a plurality of webs formed integrally with the envelope; and
the plurality of webs extend across the radial gap from the spine channel to the outer wall of the neck.

20. The balloon of claim 12, wherein the neck is arranged in coaxial alignment with the spine channel.

21. A perfusion balloon catheter, comprising at least one balloon according to claim 1.

22. The catheter of claim 21, further comprising a shaft including an inner tube and an outer tube, wherein:
the balloon is mounted on the shaft;
the inner tube of the shaft is disposed within the outer tube of the shaft;
the outer tube of the shaft is sealed to a proximal end of the balloon; and
the inner tube of the shaft extends through the balloon along the spine channel and is sealed to a distal end of the balloon.

23. The catheter of claim 22, wherein:
in a longitudinally-central portion of the expanded envelope, the at least two lobes are curved about respective longitudinal axes that are parallel to each other and to the central longitudinal axis;
the expanded envelope includes a plurality of transition portions that taper in longitudinally-outward directions from respective ends of the longitudinally-central portion of the expanded envelope;
at least one transition portion of the plurality of transition portions tapers down to a longitudinally-extending tubular neck;
the neck includes an outer wall that is an integral continuation of the envelope; and
the outer tube of the shaft is attached to the neck at the proximal end of the balloon.

24. The catheter of claim 23, wherein the inner tube of the shaft is attached to another neck disposed at the distal end of the balloon.

25. The catheter of claim 22, wherein an annulus defined between the inner tube and outer tube of the shaft is in fluid communication with the envelope.

26. The catheter of claim 22, wherein the inner tube of the shaft opens distally and defines a longitudinal conduit extending continuously through the balloon.

27. A method of making a balloon for a perfusion balloon catheter, the method comprising:
placing an elongate element into a mould cavity, the element having an internal profile that defines a spine channel around a central longitudinal axis and that extends outwardly with respect to the central longitudinal axis to join a tubular outer wall of the element at a plurality of angularly-spaced junctions; and
applying internal fluid pressure to the element to radially expand a plurality of portions of the outer wall within the mould cavity, forming at least two radially-projecting lobes that are disposed mutually spaced angularly about the central longitudinal axis;
wherein placing the element into the mould cavity includes orienting the element in the mould cavity to position the plurality of junctions at angular positions between the at least two lobes.

28. The method of claim 27, further comprising:
heating the element to a softening temperature; and
longitudinally tensioning the element;
wherein the plurality of portions of the outer wall are radially expanded when the element is under longitudinal tension and heated to the softening temperature.

29. The method of claim 27, further comprising constraining radial expansion of a plurality of portions of the outer wall that are in angular alignment with the plurality of junctions.

30. The method of claim 27, further comprising necking a plurality of end portions of the element down to a reduced diameter relative to a longitudinally central portion of the element.

31. The method of claim 30, wherein the plurality of end portions are of substantially circular cross-section and the central portion of the element has a cross-section that is radially enlarged between the plurality of junctions.

32. The method of claim 27, further comprising removing an end part of the internal profile from within the outer wall of the element.
